(19) United States
(12) Patent Application Publication
(10) Pub. No.: US 2024/0338814 A1
(43) Pub. Date: Oct. 10, 2024

(54) METHODS OF QUALITY CONTROL FOR SOFTWARE-PROCESSED ECHOCARDIOGRAM

(71) Applicant: Alpha Intelligence Manifolds, Inc., New Taipei City (TW)

(72) Inventors: YUEH-CHUN LAI, New Taipei City (TW); WIN-HOW LEE, New Taipei City (TW)

(21) Appl. No.: 18/296,963

(22) Filed: Apr. 6, 2023

(51) Int. Cl. G06T 7/00; G06T 7/13; G06V 10/774; G06V 10/776; G06V 10/82; G16H 50/20

(52) U.S. Cl. CPC G06T 7/0012; G06T 7/13; G06V 10/774; G06V 10/776; G06V 10/82; G16H 50/20; G06T 2207/10132; G06T 2207/20081; G06T 2207/20084; G06T 2207/30048; G06V 2201/03

(57) ABSTRACT

The present invention relates to a method for evaluating software-analyzed videos, comprising receiving input images and corresponding software-analyzed images, generating predicted difference parameters by at least one difference model, generating geometric parameters, and generating a predicted evaluation result based on the predicted difference parameters and the geometric parameters by an evaluation model. The present invention also relates to a method for training models to perform difference parameter generation, and a method for training models to perform evaluation result generation described above.

### BACKGROUND OF THE INVENTION

### Field of the Invention

The present invention relates to a method of quality control for software-processed images, especially for echocardiographic images.

### Description of Related Art

Medical image analysis is an important part in medical field. Recent years, artificial intelligence (AI) has applied to many medical devices and systems to analyze acquired medical images for further applications such as disease diagnosis. AIs can be trained to perform various tasks, including image analysis, to assist medical doctors in image interpretation and thus reduce the workload of medical doctors.

Echocardiography is an ultrasound of the heart, which is a type of medical imaging of the heart images routinely used in the diagnosis, management, and follow-up of patients with any suspected or known heart diseases. In recent years, some AI products are commercially available for echocardiographic image analysis. Typically, a user uploads a cardiac ultrasound video (e.g. a four-chamber view video), and the AI software will automatically fit the edge of the inner wall in the video, and use the changes of the edge in the video to calculate some parameters. However, the edges fitted by the software using statistical data models often suffer from large errors, and cannot be used directly. Thus, many of the products also provide the function of adjusting the edges on specific frames by the user after the edges are automatically fitted. In such way, an expert can instruct the software to calculate more accurate parameters by adjusting the edges.

The above workflow is quite troublesome, since for a better accuracy the expert needs to inspect every input image to check whether the automatically generated results require further adjustments. The required manual inspection for quality control contradicts the spirit of automatic fitting with AI software.

Therefore, there is a need to develop an automatic method to evaluate the quality of software-generated results.

### SUMMARY OF THE INVENTION

To resolve the problems, the present invention provides an automatic method to evaluate the accuracy of software-generated results for medical images. If the evaluation determines that the generated results are with good quality, then the user may use the results directly without further inspection. Alternatively, if the evaluation shows a bad quality, the user may decide to make manual adjustment or retake the medical image based on the evaluation summary. The method thus facilitates the diagnostic workflow for medical doctors.

One aspect of this invention provides a method of training a difference model to generate difference parameters related to the differences between a software-tracked contour and an adjusted contour, comprising training a first machine learning model with multiple first training data sets, each of the multiple first training data sets comprising a first training image set as the input for training, and a difference parameter set as the target for training. The first training image set and the difference parameter set are generated by the steps of: (a) obtaining the first training image set by selecting at least one image; (b) generating, by an analysis software, the software-tracked contour based on the first training video or the first training image set; (c) obtaining the adjusted contour; and (d) obtaining the difference parameter set based on the software-tracked contour and the adjusted contour. In one embodiment, the first training image is an echocardiographic image.

In one embodiment, each image of the first training image set is processed according to the software-tracked contour before used as the input for training.

In one embodiment, the first machine learning model is a regression model based on convolutional neural network. Specifically, the first machine learning model may be a residual neural network (ResNet) model.

Another aspect of this invention provides a method of training an evaluation model to generate predicted evaluation errors related to the differences between software-generated analysis result and adjusted analysis result, comprising training a second machine learning model with multiple second training data sets, each of the multiple second training data sets comprising at least one difference parameter set as inputs for training, at least one geometric parameter set as inputs for training, and an evaluation result as target for training. Each of the at least one difference parameter set indicates the differences between software-tracked contour and adjusted contour; each of the at least one geometric parameter set is calculated based on a software-tracked contour generated by an analysis software; and the evaluation result is determined based on the differences between a software-generated analysis result and an adjusted analysis result. In one embodiment, the second training image is an echocardiographic image.

In one embodiment, the second machine learning model is a tree-based model. Specifically, it may be a regression model, and the evaluation result may be an error value indicating the difference between the software-generated analysis result and the adjusted analysis result. Alternatively, it may also be a classification model, and the evaluation result may be a class indicating a good quality or bad quality of the software-generated analysis result.

In one embodiment, each of the at least one geometric parameter set is generated by the steps of: (a) generating, by the analysis software, a software-tracked contour from at least an image; and (b) calculating one of the at least one geometric parameter sets based on the software-tracked contour.

Each of the at least one difference parameter set may be generated by direct calculation from software-tracked contour and adjusted contour, or it may also be generated by model prediction by a difference model. In one embodiment, each of the at least one difference parameter set is generated by the steps of: (a) generating, by the analysis software, a software-tracked contour from at least one image; (b) obtaining an adjusted contour; and (c) calculating one of the at least one difference parameter set based on the software-tracked contour and the adjusted contour. In another embodiment, each of the at least one difference parameter set is generated by the steps of: (a) obtaining a second training image set by selecting at least one image; and (b) generating, by a difference model, one of the at least one difference parameter set based on the second training image set.

In one embodiment, the at least one geometric parameter set comprises an ED (end-diastolic) geometric parameter set and an ES (end-systolic) geometric parameter set. And in one embodiment, the at least one difference parameter set comprises an ED (end-diastolic) difference parameter set and an ES (end-systolic) difference parameter set.

In one embodiment, the evaluation result is generated by the steps of: (a) calculating a software-generated analysis result based on the tracked ED contour and the tracked ES contour; (b) obtaining an adjusted ED contour and an adjusted ES contour; (c) calculating an adjusted analysis result based on the adjusted ED contour and the adjusted ES contour; and (d) determining the evaluation result based on the software-generated analysis result and the adjusted analysis result.

In yet another aspect, the present invention provides a method of quality control for software-analyzed images, comprising: (a) receiving at least one input image and at least one corresponding software-analyzed image, wherein the at least one corresponding software-analyzed image is generated by analyzing the at least one input image with an analysis software; (b) generating, by at least one difference model, at least one set of predicted difference parameters based on the at least one input image; (c) generating at least one set of geometric parameters from the at least one corresponding software-analyzed image; and (d) generating, by an evaluation model, a predicted evaluation result based on the at least one set of predicted difference parameters and the at least one set of geometric parameters.

In one embodiment of the quality control method, the evaluation result is an error value indicating the difference between a software-generated analysis result and an adjusted analysis result. In another embodiment, the evaluation result is a class indicating a good quality or bad quality of the software-generated analysis result.

The present invention also provides a non-transitory computer-readable medium having stored thereon a set of instructions that are executable by a processor of a computer system to carry out a method of: (a) receiving at least one input image and at least one corresponding software-analyzed image, wherein the at least one corresponding software-analyzed image is generated by analyzing the at least one input image with an analysis software; (b) generating, by at least one difference model, at least one set of predicted difference parameters based on the at least one input image; (c) generating at least one set of geometric parameters from the at least one corresponding software-analyzed image; and (d) generating, by an evaluation model, a predicted evaluation result based on the at least one set of predicted difference parameters and the at least one set of geometric parameters.

Other objectives, advantages and novel features of the invention will become more apparent from the following detailed description when taken in conjunction with the accompanying drawings.

### BRIEF DESCRIPTION OF THE DRAWINGS

FIG. 1 is the workflow of training the first model (a difference model).

FIG. 2 is the workflow of training the second model (an evaluation model).

FIG. 3 is the workflow of the quality control for software-processed images.

FIG. 4A is the echocardiogram video on end-diastolic (ED) frame with endomyocardium contour tracked by an analysis software. FIG. 4B is the echocardiogram video on end-systolic(ES) frame with endomyocardium contour tracked by an analysis software. FIG. 4C is the echocardiogram video on end-diastolic (ED) frame with endomyocardium contour adjusted by an expert. FIG. 4D is the echocardiogram video on end-systolic(ES) frame with endomyocardium contour adjusted by an expert.

FIG. 5A is the extracted contour from the end-diastolic (ED) frame of FIG. 4A, and FIG. 5B is the area of the contour.

FIG. 6 shows some points assigned to the contour and/or the area. Points A, M, L, R and C are defined in this figure.

FIG. 7A shows the frame of the original echocardiographic video to be applied with the mask. FIG. 7B shows a mask obtained by distance transformation and decay performed linearly from the edge of extracted contour (FIG. 5A) or the area (FIG. 5B). FIG. 7C shows the masked region of FIG. 7A, and FIG. 7D shows the image cropped into smaller size.

FIGS. 8A-8D show the test results predicted by the ES frame difference model. The horizontal axis represents the ground truth values, and the vertical axis represents the predicted values. FIG. 8A is the Dice score of left ventricle endocardium area, FIG. 8B is the area difference between automated and predicted manual results, FIG. 8C is the difference of line LR y intercept, and FIG. 8D is Dynamic Time Warping (DTW) distance between auto and manual endocardium contour.

FIGS. 9A-9D show the test results predicted by the ED frame difference model. The horizontal axis represents the ground truth values, and the vertical axis represents the predicted values. FIG. 9A is the Dice score of left ventricle endocardium area, FIG. 9B is the area difference between automated and predicted manual results, FIG. 9C is the difference of line LR y intercept, and FIG. 9D is Dynamic Time Warping (DTW) distance between auto and manual endocardium contour.

FIGS. 10A and 10B show the test results predicted by the evaluation model. FIG. 10A shows the ground truth and the predicted values of GLS difference, and FIG. 10B is the ROC curve of QC pass/fail for the model. The R-squared value of FIG. 10A is 0.4499, the mean absolute error is 1.5586, and the QC pass/fail accuracy is 74.18% (with cutting off at GLS difference=2).

FIGS. 11A-11D show an example of bad tracking result caused by bad image quality. FIG. 11A is the automatically tracked ED frame from an echocardiogram video, FIG. 11B is the automatically tracked ES frame, FIG. 11C is the manually adjusted ED frame, and FIG. 11D is the manually adjusted ES frame.

FIGS. 12A-12D show another example of bad tracking result caused by bad image quality. FIG. 12A is the automatically tracked ED frame from an echocardiogram video, FIG. 12B is the automatically tracked ES frame, FIG. 12C is the manually adjusted ED frame, and FIG. 12D is the manually adjusted ES frame.

FIGS. 13A-13D show an example of bad tracking result caused by bad software analysis. FIG. 13A is the automatically tracked ED frame from an echocardiogram video, FIG. 13B is the automatically tracked ES frame, FIG. 13C is the manually adjusted ED frame, and FIG. 13D is the manually adjusted ES frame.

FIGS. 14A and 14B show the test results predicted by a linear regression model. FIG. 14A shows the ground truth and the predicted values of GLS difference, and FIG. 14B is the ROC curve of QC pass/fail for the model. The R-squared value of FIG. 14A is 0.03, the mean absolute error is 1.7731, and the QC pass/fail accuracy is 58.38% (with cutting off at GLS difference=2).

FIG. 15 shows the test results predicted by a model trained directly with whole images (without geometric parameters). The R-squared value is 0.1438, and the mean absolute error is 2.1109.

### DETAILED DESCRIPTION OF PREFERRED EMBODIMENTS

The terminology used in the description presented below is intended to be interpreted in its broadest reasonable manner, even though it is used in conjunction with a detailed description of certain specific embodiments of the technology. Certain terms may even be emphasized below; however, any terminology intended to be interpreted in any restricted manner will be specifically defined as such in this Detailed Description section.

The embodiments introduced below can be implemented by programmable circuitry programmed or configured by software and/or firmware, or entirely by special-purpose circuitry, or in a combination of such forms. Such special-purpose circuitry (if any) can be in the form of, for example, one or more application-specific integrated circuits (ASICs), programmable logic devices (PLDs), field-programmable gate arrays (FPGAs), graphics processing units (GPUs), etc.

The method to establish the quality evaluation system comprises four steps, including data labeling, image preprocessing, model training, and inference pipeline. In the present invention, two kinds of machine learning models are constructed to perform the work of quality control (QC) for software-processed/analyzed images. After training, the first model may use an input (unprocessed) image to predict some difference parameters, and the second model may use the difference parameters and geometric parameters derived from the software-processed/analyzed image to evaluate the quality of software-processing and/or analysis.

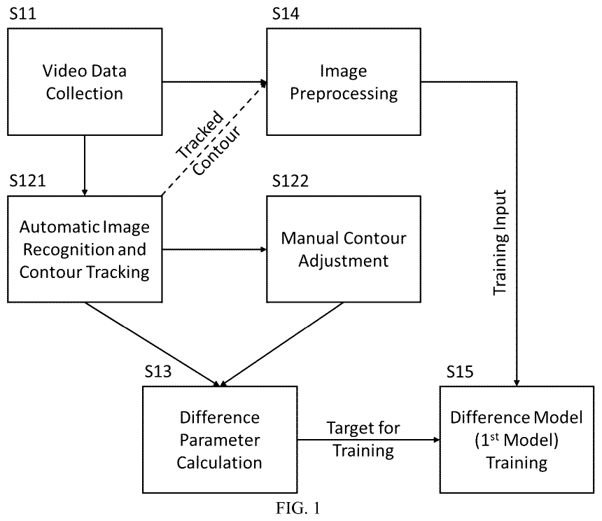

FIG. 1 shows the training steps of the first model, which is a difference model. Step S11 is data collection, where multiple videos (e.g. echocardiographic image series) are collected as the training dataset. In Step S121, training data are input into the automatic analysis software, and the analysis and calculation results along with the image tracking results are recorded. An example of automatic analysis is finding specific image frames from an input video by the software, such as finding end-diastolic and end-systolic frames from an echocardiographic video. The tracking then may be applied to those selected frame(s), and the tracking result may be a contour automatically tracked by the analysis software. Then in Step S122 an expert adjusts the automated image tracking results to more accurate ones, such as redraw a corrected contour. In Step S13 the automatically tracked and adjusted results are recorded, and the difference parameters which describe differences between automatically tracked and adjusted results are calculated.

In Step S14, before model training, the input images, which are the selected video frames, may be preprocessed to make the subsequent model focus on key image areas and incorporate the automated tracking results of the software. For example, the automated tracking result may be used to determine regions of masking and cropping. The input image may then be masked and cropped, leaving the key tracking area in the image.

As described above, the model training in the present invention is divided into two parts. The first part is a difference model using a neural network to predict difference parameters, wherein the difference parameters indicate the difference between software-generated and expert adjusted results (e.g. software-tracked contour and adjusted contour). The first step is to calculate the parameters describing the difference between the image tracking results before and after the adjustment by experts, wherein some meaningful parameters are specifically selected by experts. As shown in Step S15 in FIG. 1, with the difference parameters obtained in Step S13 used as the target to learn, and with the preprocessed image obtained in Step S14 used as the input, a regression neural network model is trained to output the value of each parameter.

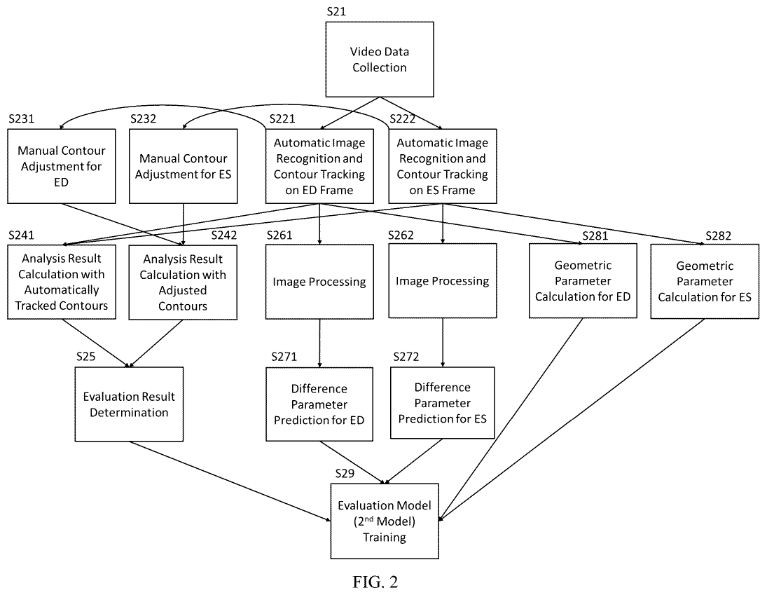

The second part of model training is to train a tree-based evaluation model to predict the difference between the final analysis value (e.g. global longitudinal strain) of the automatic analysis software and the value adjusted by experts. The input are (1) the difference parameters predicted in the first part by the difference model, and (2) the geometric parameter directly measured and quantified in the automatic tracking result of the software. The output of the second model predicts the error between the results originally generated by the analysis software and that adjusted by experts. A threshold value may be set to evaluate the quality of automatic tracking and analysis results. If the predicted difference is less than the threshold value, a user (e.g. a medical doctor) may trust the automatic analysis results. FIG. 2 shows the training steps of the second model.

In Step S21, multiple videos (e.g. echocardiographic image series) are collected as the training dataset. The dataset may be the same as the one used to train the first model (as described in Step S11), or it may be a different dataset. In Steps S221 and S222, two image sets, ED set and ES set, are separately tracked by the automatic analysis software. The procedure is similar to S121. In Steps S231 and S232, the tracked ED and ES contours are separately adjusted, which is similar to Step 122. In Step S241, the automatically tracked ED and ES contours obtained in Steps S221 and S222 are integrated to evaluate an automatic analysis result, such as calculating the global longitudinal strain (GLS) value based on the software-tracked ED and ES contours. The analysis result is the same as what the automatic analysis software outputs without contour adjustment by an expert. In Step S242, the manually adjusted ED and ES contours obtained in Steps S231 and S232 are integrated to evaluate an expert-adjusted analysis result, such as calculating the global longitudinal strain (GLS) value based on the adjusted ED and ES contours. The analysis result is the same as what the automatic analysis software outputs with contour adjustment by an expert. In Step S25, the analysis result generated in Steps S241 and S242 are combined to calculate an analysis error, which represents the error caused by automatic analysis software (compared to the expert-adjusted result).

In Steps S261 and S262, the two image sets obtained in Steps S221 and S222 may be independently preprocessed, which is similar to Step S14. The independently preprocessed images then are used to generate difference parameters for ED and ES, as shown in Steps S271 and S272. The difference parameters for ED and ES are the parameters describing the differences between software-generated and adjusted contours for ED and ES frames. The difference parameters may be generated (predicted) by established difference models using preprocessed images as inputs. Alternatively, it may also be generated (calculated) by obtaining software-tracked ED and adjusted ED (and for ES frame, by obtaining software-tracked ES and adjusted ES) and calculating those parameters. In Steps S281 and S282, geometric parameters for ED and ES, respectively, are calculated based on the automatically tracked ED and ES contours obtained in Steps S221 and S222. The geometric parameters represent the geometric properties of the software-tracked contours.

In Step S29, the data obtained in Steps S25, S271, S272, S281, S282 are used to train the second model, which is an evaluation model. The difference parameters obtained in Steps S271 and S272, and the geometric parameters obtained in Steps S281 and S282 are used as the input for training. The evaluation error values obtained in Step S25 are used as the learning target for training.

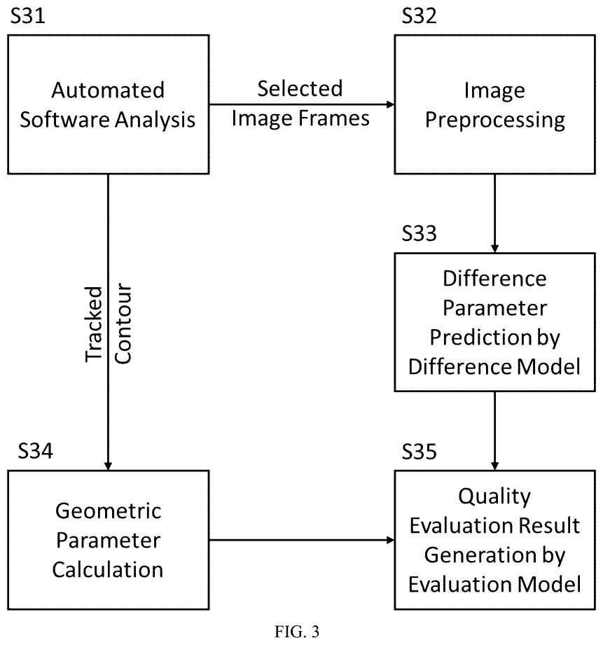

In the inference pipeline step, the trained model, the preprocessed images, and the analysis software are arranged into a pipeline, as shown in FIG. 3. In Step S31, an input video (e.g. an echocardiogram video) is processed and analyzed by an analysis software to perform automated analysis. The outputs, which may include key frames in the video, the tracked contours, and the analysis result, are used in QC evaluation. In Step S32, the extracted frames (e.g. frames near ED frame and ES frame) optionally undergo image preprocessing based on the software-tracked contours to minimize background interference. An example of image preprocessing is to crop the background out from the images and retain only the contour region. In Step S33, the processed image frames are then analyzed by the difference model to predict difference parameters between automated tracked and expert adjusted contours. In a preferred embodiment, two difference models are used to independently generate predicted difference parameters for ED contour and ES contour. In Step S34, several geometric parameters are calculated based on the software-tracked contour(s). The parameters are some measurements of the contour. In Step S35, the difference parameters (predicted by the difference model in Step S33) and the geometric parameters (calculated in Step S34) are used as the inputs for the evaluation model, the second trained model. The evaluation model then generates an evaluation indicating the quality of analysis result performed by automated software. The quality of the results generated by the software can thus be predicted from an input video, such as an echocardiogram.

The following provide more details for each step.

### Data Labeling

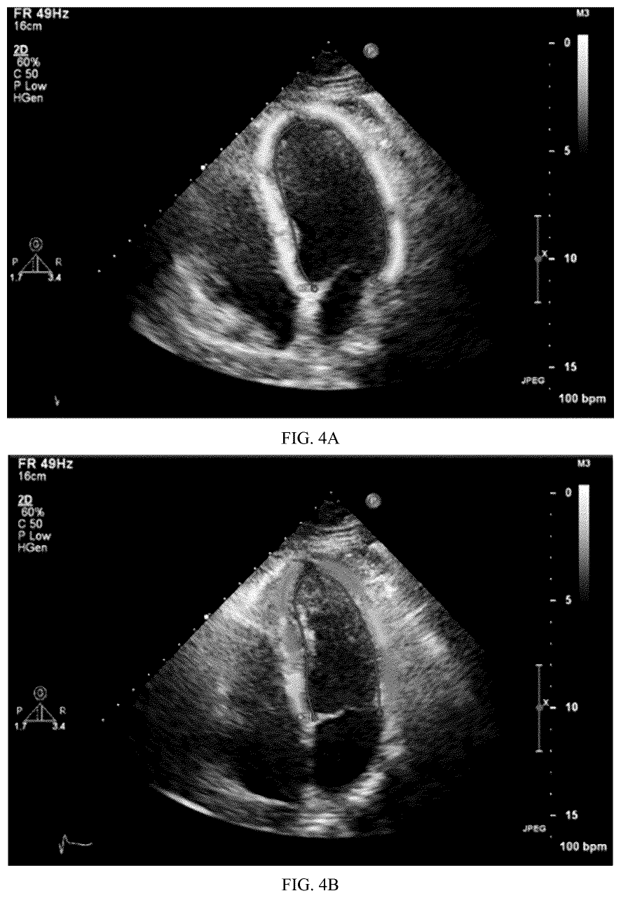

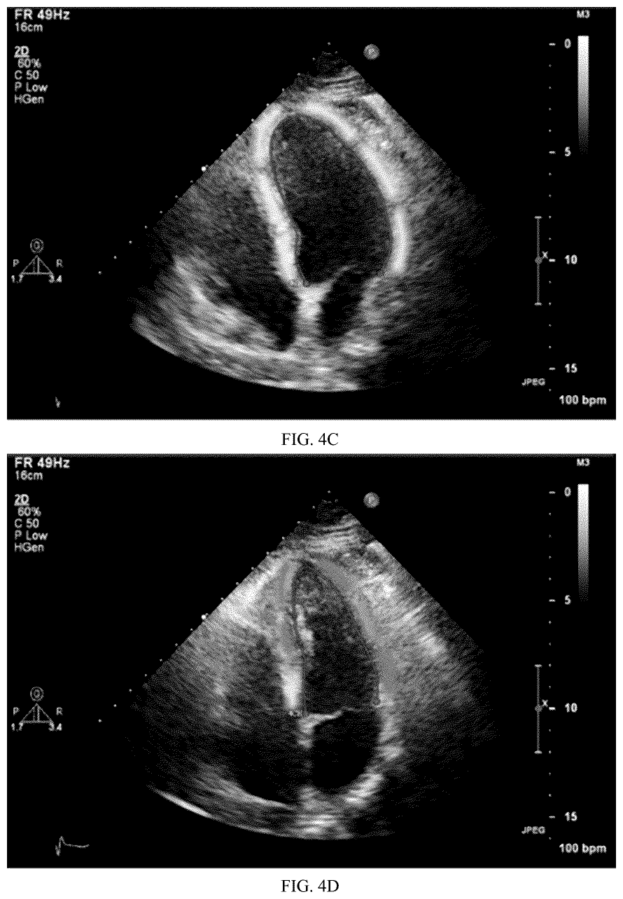

Echocardiogram videos in database are collected for data labeling. In data labeling, each of the input echocardiogram videos is sent to a software (e.g. Tomtec AutoStrain) for analysis, and the automated global longitudinal strain (GLS) analysis result and the myocardial contour tracking results (e.g. a tracked endomyocardium contours) of left ventricle are collected. The GLS analysis result is numeric, and the myocardial contour tracking results may be captured as screenshot images or directly exported as coordinates of keypoints on end-diastolic (ED) and end-systolic(ES) frames, as shown in FIG. 4A and FIG. 4B. Then an expert may adjust the myocardial tracking result in the software to get a more precise GLS analysis result, as shown in FIG. 4C and FIG. 4D. The adjusted GLS analysis result and corresponding adjusted myocardial contour are then also collected for further use. The data obtained by the above echocardiogram labeling steps may include:
(1) Automated myocardial contour tracking result on ED frame,
(2) Automated myocardial contour tracking result on ES frame,
(3) Manual adjusted myocardial contour on ED frame,
(4) Manual adjusted myocardial contour on ES frame,
(5) Automated global longitudinal strain, and
(6) Manual adjusted global longitudinal strain.

### Geometric and Difference Parameter Calculation

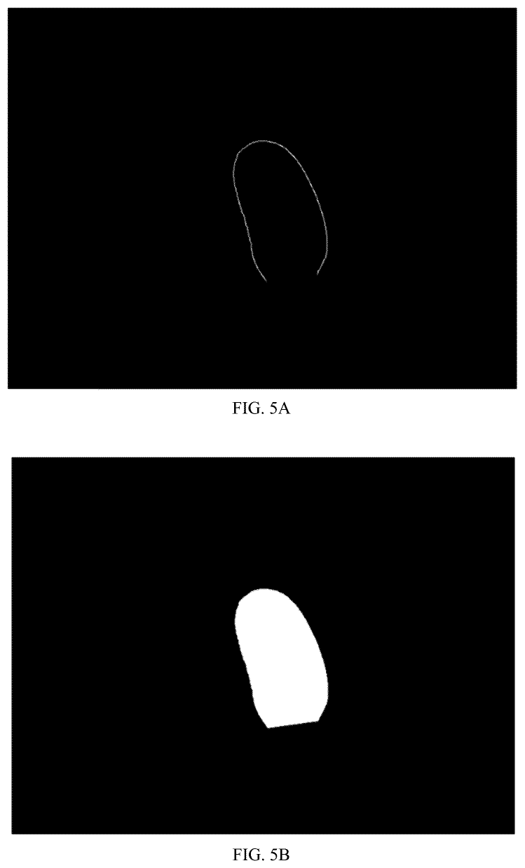

In geometric parameter calculation, the tracked endomyocardium contour and left ventricle area from the tracking result image are extracted. FIG. 5A is an example of the extracted contour from the end-diastolic (ED) frame of FIG. 4A, and FIG. 5B is the area of the contour. Some points may be assigned to the contour and/or the area, as shown in FIG. 6. Based on the contour, the enclosed area, and the assigned points, some geometric metrics of automated tracking result are measured, which may include:
(1) Area of the contour,
(2) Slope and y intercept of line LR,
(3) Slope and y intercept of line AM,
(4) Basal width (length of LR segment),
(5) Height (length of AM segment), and
(6) Height width ratio.

Another contour and left ventricle area can be extracted from the expert-adjusted contour of endomyocardium. Then the difference of the contours and areas between automated and manual adjusted contours may be calculated as additional parameters, which may include:
(1) Difference of line LR slope,
(2) Difference of line AM slope,
(3) Difference of line AM y intercept,
(4) Distance between A point of auto and manual contours,
(5) Distance between L point of auto and manual contours,
(6) Distance between R point of auto and manual contours,
(7) Distance between M point of auto and manual contours,
(8) Distance between C point of auto and manual contours,
(9) Distance between auto and manual endocardium right side contour segment,
(10) Distance between auto and manual endocardium apex contour segment,
(11) Distance between auto and manual endocardium left side contour segment,
(12) 2D distance between auto and manual endocardium right side contour segment,
(13) 2D distance between auto and manual endocardium apex contour segment,
(14) 2D distance between auto and manual endocardium left side contour segment,
(15) Dice score between auto and manual areas,
(16) Area difference,
(17) Dynamic time warping distance between auto and manual contour,
(18) Slope and y intercept difference between auto and manual line LR,
(19) Slope and y intercept difference between auto and manual line AM, and
(20) Point distance between auto and manual L, R, C, A points.

Some or all of the parameters may be selected to express the quality of endomyocardium contour tracking result. The selected parameters are calculated for both tracking results of ED frame and ES frame, and trained separately in later steps.

### Image Preprocessing

This step is optional but may accelerate the training speed of the AI and the execution speed of the trained model. Although the input echocardiogram is a video, the training of the models usually does not require the whole video as the input. As a result, the video frames near end-diastolic (ED) and end-systolic(ES) frames may be selected as input images, since the parameters are measured and calculated from ED and ES frames.

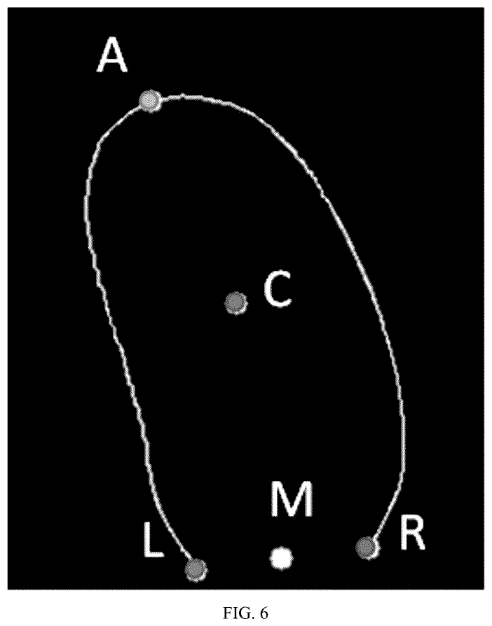

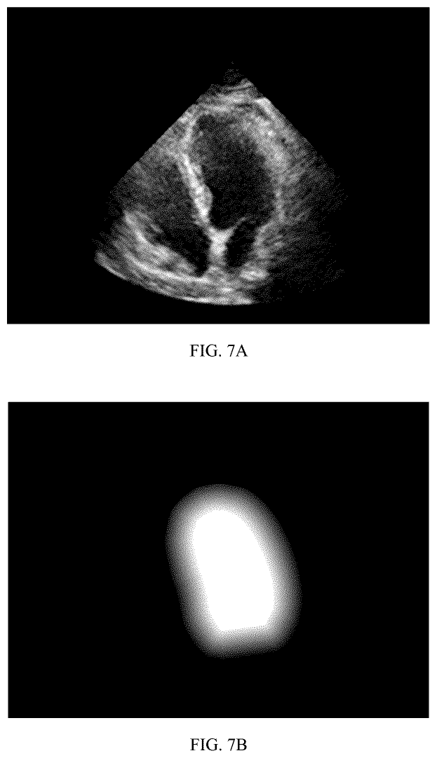

Based on the extracted left ventricle area from the screenshot of automated tracking contour, a mask may be created to facilitate the deep neural network model focusing on relevant image area. The mask generation, application, and image cropping are as shown in FIGS. 7B-7D.

### Model Training

This step is to train a model to evaluate the quality of automatically generated analysis result. The model may be divided into two parts. The first part is a difference model, and the second part is an evaluation model.

### (1) Difference Model Training

In the first part, using the preprocessed image frames and the difference parameters as training data, two regression neural networks may be constructed to predict the difference parameters which describe the differences between automated tracking and manual adjusted results (e.g. software-tracked contour and adjusted contour). One neural network takes one or more preprocessed image frames around ED as input, and outputs the difference parameters calculated from ED contour tracking result. The other neural network does the same but uses images and parameters of ES instead of ED. The model may use one preprocessed image frame to generate a satisfactory contour tracking result, or it may use a plurality of preprocessed image frames as input to reduce the noise in a single frame.

### (2) Evaluation Model Training

The above predicted difference parameters (predicted by the difference model) and measured geometric parameters (calculated from the automated tracking contours) may be combined to train an evaluation model to predict the final target: the difference between automated GLS and expert adjusted GLS (and thus the quality of automated tracking and analysis result). The geometric parameters can be automatically measured from automated tracking contour when inferencing new data.

The evaluation model may be a classifier (a classification model) to determine if the difference is large (implying bad tracking and analysis result of the automated software) or small (implying good tracking and analysis result of the automated software), or may be a regressor (a regression model) to tell exactly how much the GLS difference is between the software-generated analysis result and the expert adjusted analysis result.

### EXAMPLES

The following examples are provided to further illustrate the details of training models for quality control of software-processed/analyzed echocardiographic images.

### 1. Data Collection and Labeling

Around 1000 apical four-chamber view echocardiographic videos are used as raw data. The raw data are split into training/validation/testing sets. Splitting strategy ensured data with good tracking and bad tracking results are evenly distributed across datasets. Two sets of measurements/labels: Auto GLS and Manual GLS are generated using these datasets. Auto GLS are generated by using Tomtec (TOMTEC Imaging Systems GmbH) AutoStrain software with automatic endocardium contour tracking and GLS computing. Manual GLS labels are generated from contours defined by medical doctors.

### 2. Geometric Parameter Calculation

After data labeling, geometric parameters measured from automatic tracked endocardium contour are generated for each echocardiographic video. Referring to the points defined in FIG. 6, the following parameters are calculated:
(1) Area of left ventricle endocardium (A)
(2) Slope of line LR
(3) Y intercept of line LR
(4) Slope of line AM
(5) Y intercept of line AM
(6) Height of left ventricle endocardium (H)
(7) Basal width of left ventricle endocardium (B)
(8) Ratio of H/B
(9) Ratio of A/(H*B)

Based on the above parameters, difference parameters could be calculated for software-tracked contours and adjusted contours, which includes:
(1) Difference of line LR slope,
(2) Difference of line AM slope,
(3) Difference of line AM y intercept,
(4) Distance between A point of auto and manual contours,
(5) Distance between L point of auto and manual contours,
(6) Distance between R point of auto and manual contours,
(7) Distance between M point of auto and manual contours,
(8) Distance between C point of auto and manual contours,
(9) Distance between auto and manual endocardium right side contour segment,
(10) Distance between auto and manual endocardium apex contour segment,
(11) Distance between auto and manual endocardium left side contour segment,
(12) 2D distance between auto and manual endocardium right side contour segment,
(13) 2D distance between auto and manual endocardium apex contour segment,
(14) 2D distance between auto and manual endocardium left side contour segment,
(15) Dice score between auto and manual areas,
(16) Area difference,
(17) Dynamic time warping distance between auto and manual contour,
(18) Slope and y intercept difference between auto and manual line LR,
(19) Slope and y intercept difference between auto and manual line AM, and
(20) Point distance between auto and manual L, R, C, A points.

### 3. Image Preprocessing

For an input echocardiographic image (FIG. 7A), software-tracked left ventricle endocardium contour and the corresponding area are taken, as shown in FIG. 5A and FIG. 5B. Then a distance transformation are performed to obtain a mask, as shown in FIG. 7B. The mask is then applied to the frames of the input echocardiographic video (FIG. 7A) to retain only the region enclosed by left ventricle endocardium, as shown in FIG. 7C. The masked images are then cropped to eliminate excess background area, as shown in FIG. 7D.

### 4. Difference Model Training

Two regression neural networks are trained to predict the parameters which describe the differences between automated tracking and manual adjusted results. One neural network takes preprocessed image frames near ED frame as input, and outputs the difference parameters of the ED frame. The other neural network does the same but uses images and parameters of ES instead of ED. The frames near ED and ES are extracted as training images. For ED model training, 4 echocardiographic video around ED are extracted. For ES model training, 8 echocardiographic video frames around ES are extracted.

The next step after image extraction is data augmentation. Each image is shifted, scaled, and applied random brightness/contrast adjustment.

A deep residual learning model, ResnetRS3D-50 (arXiv: 2103.07579, model code: https://github.com/tensorflow/models) is used to train the difference model.

The inputs for the ED and ES difference models are 4 and 8 frames. The difference parameters calculated from training set data are used as the learning target (ground truth). Before model training, the values of the parameters are standardized. The output layer is a dense layer to output continuous values of difference parameters as listed above in Geometric Parameter Calculation paragraph. The models are trained to predict the difference parameters of echocardiographic video with associated software-tracked endocardium contour.

The models are trained under Tensorflow 2.9.1 environment with Nvidia RTX A6000 GPU for 100 epochs.

### 5. Training Result of Difference Model

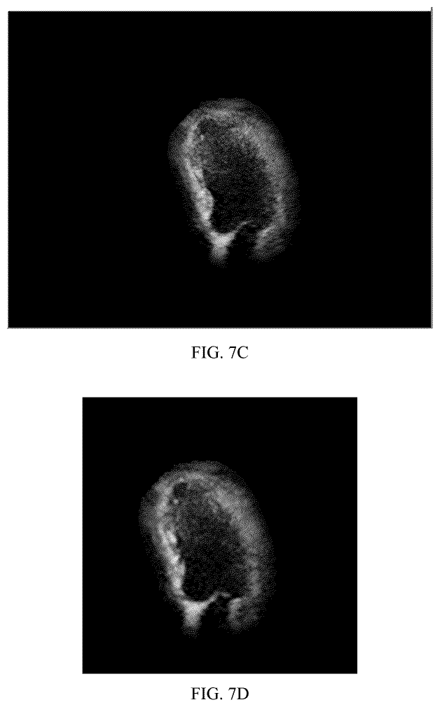

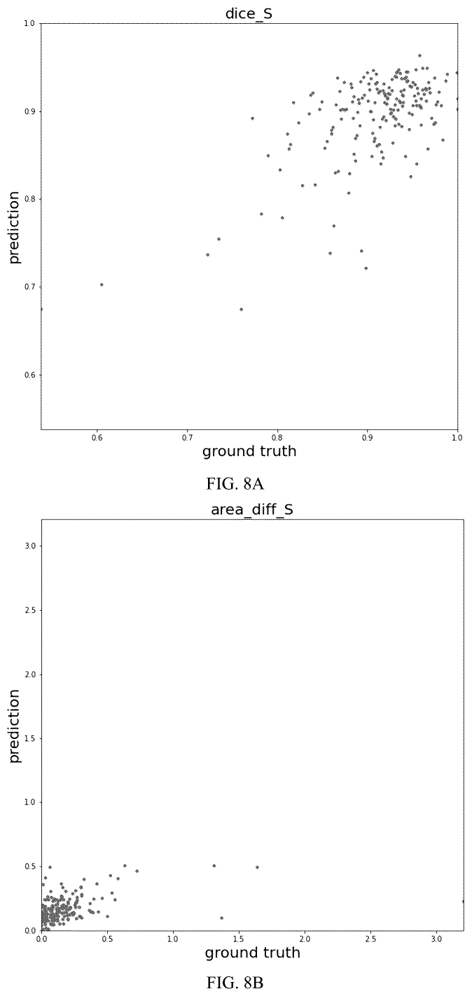

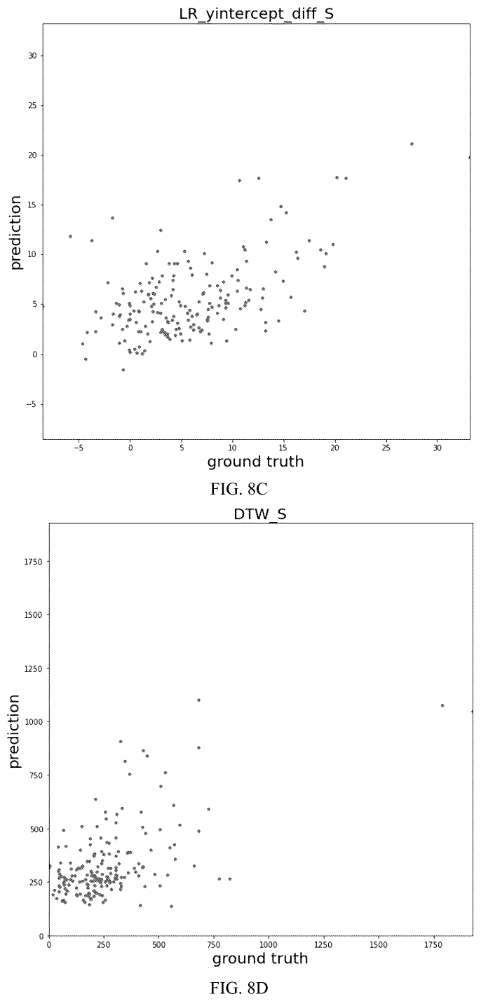

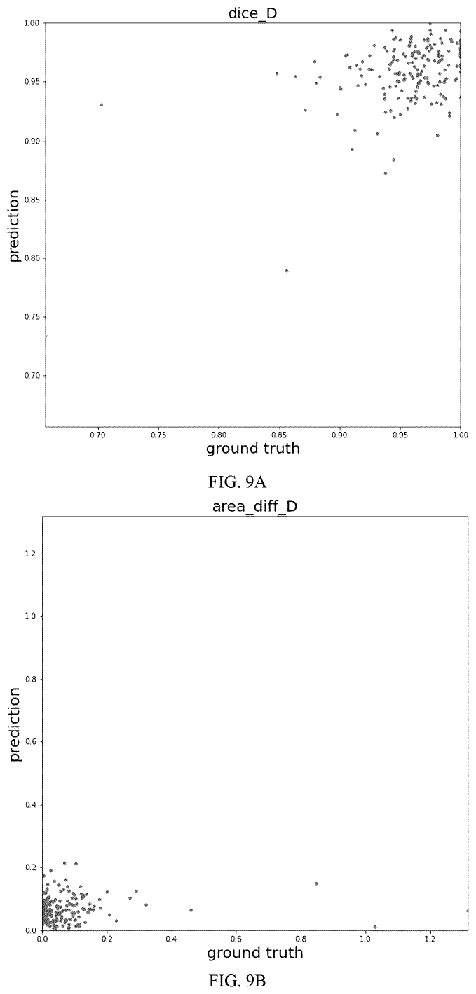

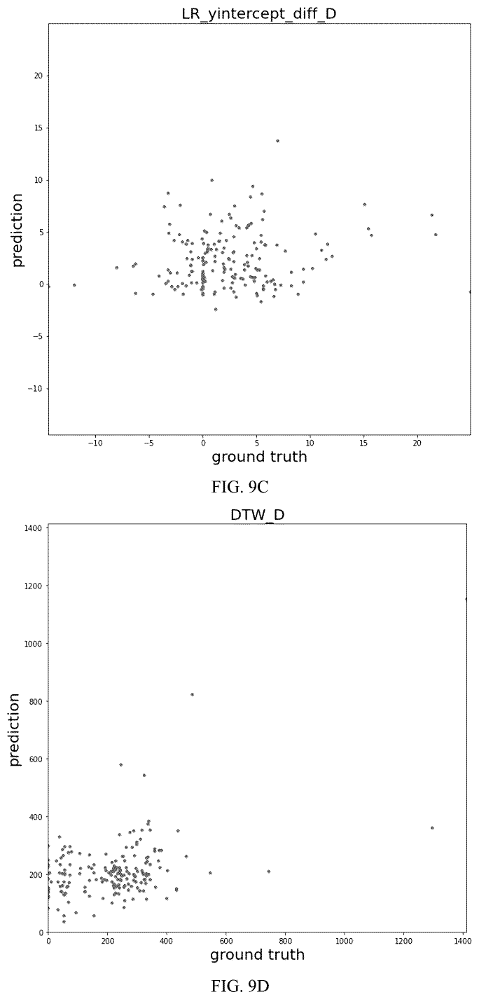

The training results of difference models are tested by the test dataset. The test results of ES frame difference model are shown in FIGS. 8A-8E. The total mean average error is 0.5931 (of variance). The test results of ED frame difference model are shown in FIGS. 9A-9E. The total mean average error is 0.7253 (of variance).

### 6. Evaluation Model Training

The results predicted by ES frame difference model and ED frame difference model are used to train an evaluation model. A tree-based model, XGBoost (https://github.com/dmlc/xgboost) algorithm, is used in training. The GLS difference calculated from the labeled data (comprising software-tracked contours and adjusted contours) are used as training ground truth. The input contains the geometric and difference parameters described above in Geometric Parameter Calculation paragraph, and the output is the error value, which is the difference between Manual GLS and Auto GLS.

### 7. Training Result of Evaluation Model

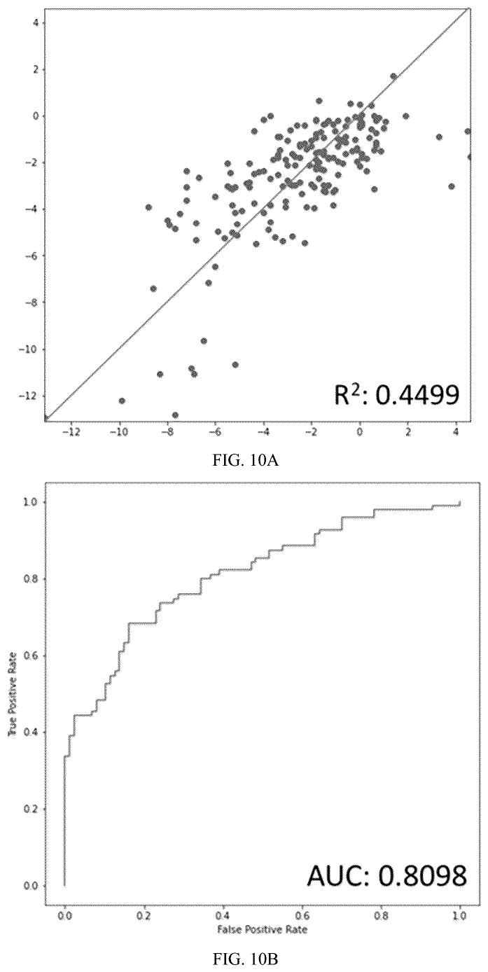

The trained evaluation model is tested by the test dataset, as shown in FIG. 10A and FIG. 10B. The mean absolute error is 1.5586, and the QC pass/fail accuracy is 74.18% (with cutting off at GLS error value=2, since the experts suggest that a GLS error over 2 is considered bad auto tracking result).

8. Bad Image Quality vs. Bad Image Analysis

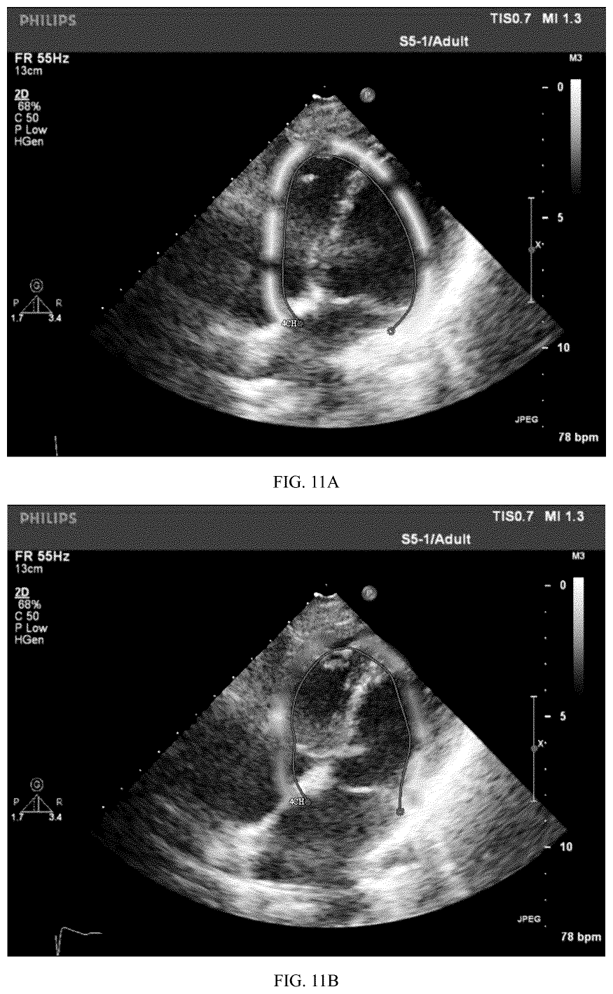

Bad prediction result generated by a software might arise from (1) bad image quality (e.g. low resolution or wrong shooting angle) or (2) good image quality but bad analysis result predicted by the automatic analysis software. The present invention can deal with both cases, as shown in FIG. 11-FIG. 13.

FIGS. 11A-11D shows an input with bad image quality, for some of the left ventricle area is not properly acquired in the input image. This causes an error to the automatically tracked GLS value. The automatically tracked GLS value is −11.2%, and the manually adjusted GLS is −18.7%. The error value (manual minus auto) is −7.5, which is much less than−2 and classified as bad prediction result. The model in the present invention predicts that the error value is −10.38, which is also much less than −2 and thus classified as bad prediction result.

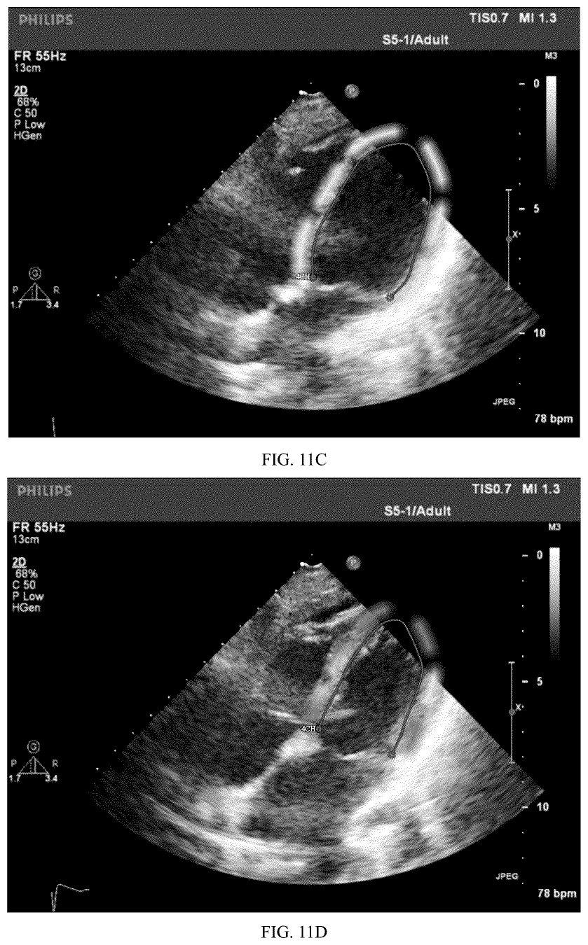

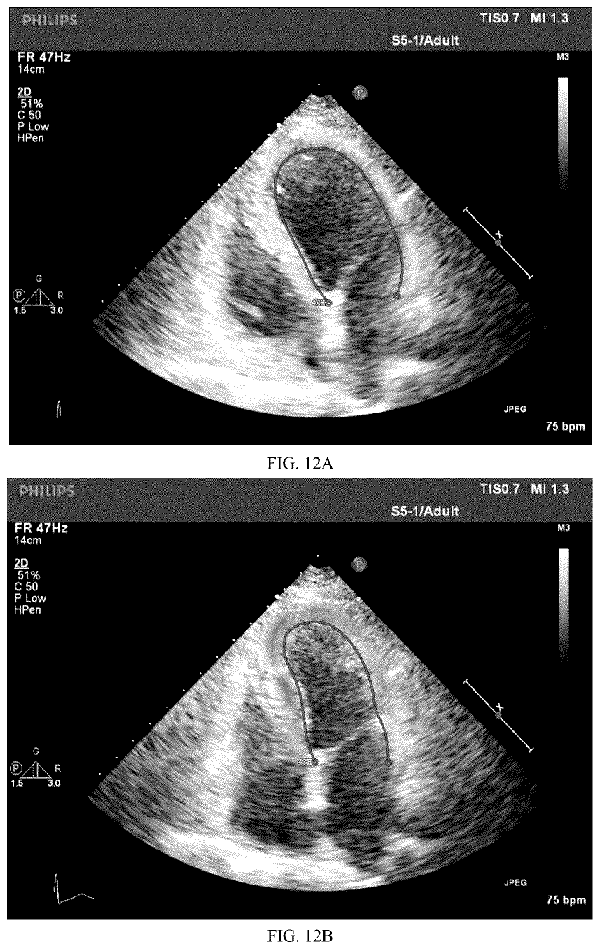

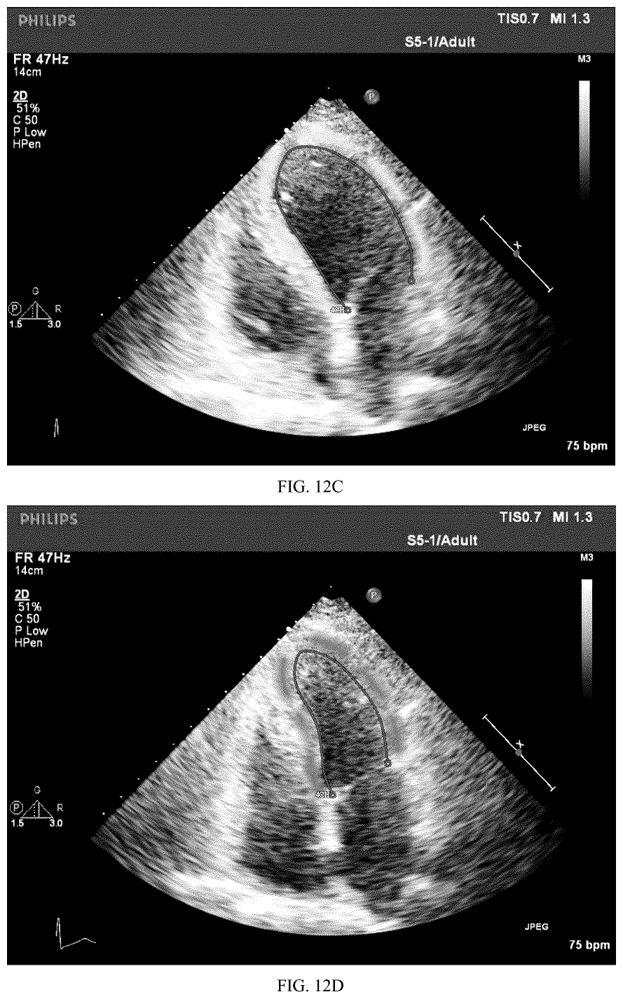

FIGS. 12A-12D shows another example of input with bad image quality. The brightness of the image is too high, which causes an error to the automatically tracked GLS value. The automatically tracked GLS value is −12.3%, and the manually adjusted GLS is −20.9%. The error value (manual minus auto) is −8.6, which is much less than −2 and classified as bad prediction result. The model in the present invention predicts that the error value is −7.42, which is also much less than −2 and thus classified as bad prediction result.

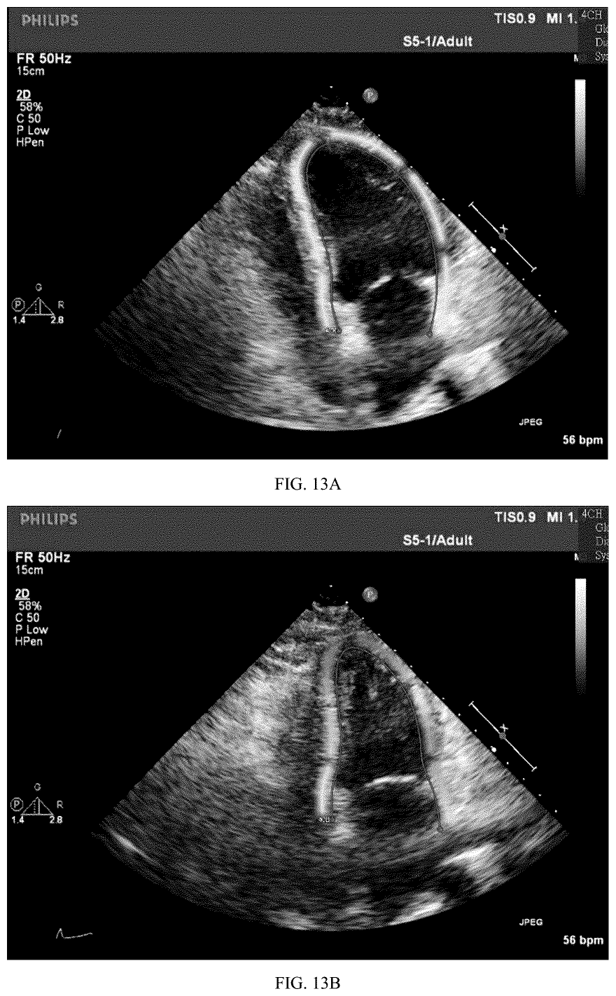

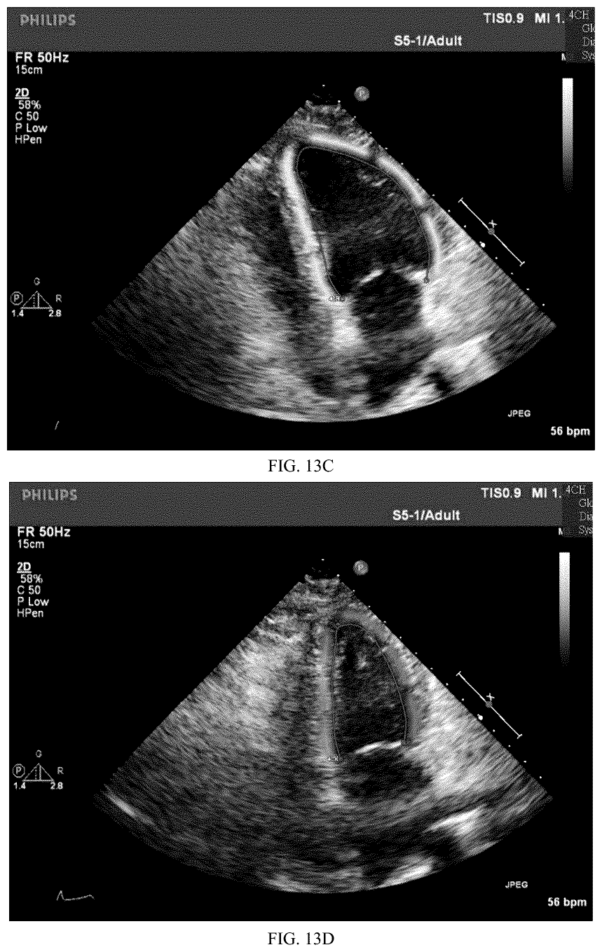

FIG. 13A-13D shows an input with fine image quality, but with bad automatic analysis. The automatically tracked GLS value is −12.3%, and the manually adjusted GLS is −22.2%. The error value (manual minus auto) is −9.9, which is much less than −2 and classified as bad prediction result. The model in the present invention predicts that the error value is −12.21, which is also much less than −2 and thus classified as bad prediction result.

### 9. Comparison With Previous Methods

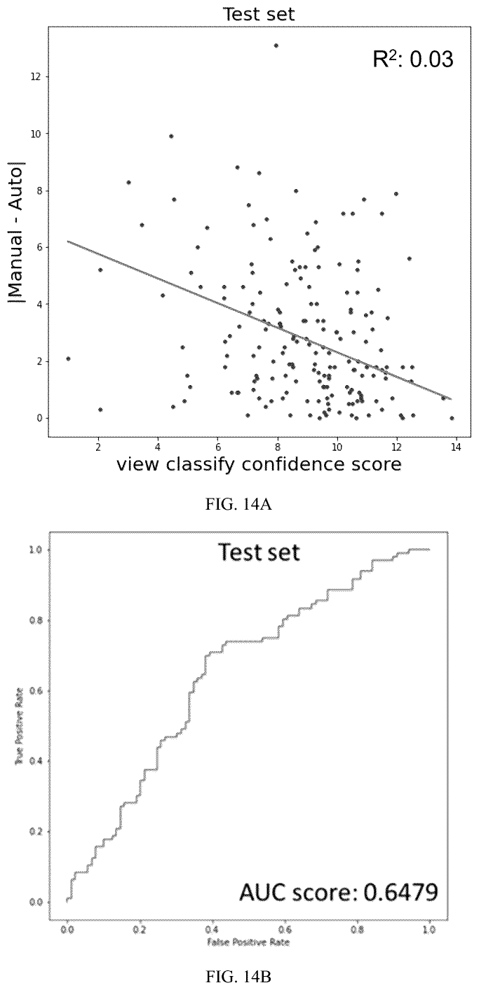

The performance of the models is compared with the a previously available model, which is a view classifier with confidence score correlates to GLS error value between automated and expert adjusted results. A simple linear regression model (since there is only one input feature, i.e. the view classifier confidence score) is employed and the same training dataset are used to train the model. The trained model is then tested by the test dataset. The result (FIGS. 14A and 14B) shows a mean absolute error of 1.7731, with the QC pass/fail accuracy of 58.92% (with cutting off at GLS error value=2).

Many prior researches indicated that the higher view classifier confidence score, the better image quality, and the closer between automatic GLS and manual adjusted GLS. The correlation, however, is very weak. Using the same test dataset, the manual-auto GLS difference predicted by our model has a R-squared value of 0.4499 to the ground truth GLS error value. On the other hand, the view classifier confidence score has a R-squared value of only 0.03 to the ground truth GLS error value.

### 10. Comparison of Model Training With Geometric Parameters and Model Training With Whole Images

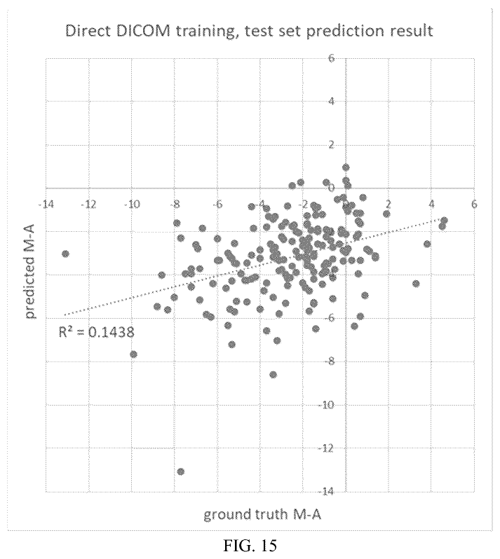

Lastly, the performance of the models trained with geometric parameters and the models trained directly with whole images (without introducing geometric parameters) are compared. For comparison, a ResnetRS3D-50 is used to construct a model to predict the error value between Manual GLS and Automatic GLS directly (without predicting difference parameters and calculating geometric parameters first). The same training dataset are used to train the model. The training input is the sampled 16 frames from one cardiac cycle in the DICOM video. The models are trained under Tensorflow 2.9.1 environment with Nvidia RTX A6000 GPU for 100 epochs. The result (FIG. 15) shows a mean absolute error of 2.1109, with the QC pass/fail accuracy of 58.38% (with cutting off at GLS error value=2).

The above result shows that training a GLS error evaluation model directly from DICOM image without introducing the geometric parameters results in a higher prediction error and a lower QC pass/fail accuracy. Also, this directed trained model acts more like a black box as it could not tell if the input image pass/failed the quality check because of which geometric features as clue. The method of the present invention, which predicts the quality check via the geometric parameters, is more accurate and makes more sense to cardiac experts.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein but is to be accorded the widest scope consistent with the principles and novel features disclosed herein. It is contemplated that additional embodiments are within the spirit and true scope of the disclosed subject matter. Thus, it is intended that the present invention covers modifications and variations that come within the scope of the appended claims and their equivalents.

What is claimed is:

1. A method of training a difference model to generate difference parameters related to the differences between a software-tracked contour and an adjusted contour, comprising training a first machine learning model with multiple first training data sets, each of the multiple first training data sets comprising a first training image set as the input for training, and a difference parameter set as the target for training, wherein the first training image set and the difference parameter set are generated by the steps of:
(a) obtaining the first training image set by selecting at least one image;
(b) generating, by an analysis software, the software-tracked contour based on the first training video or the first training image set;
(c) obtaining the adjusted contour; and
(d) obtaining the difference parameter set based on the software-tracked contour and the adjusted contour.

2. The method of claim 1, wherein the first training image is an echocardiographic image.

3. The method of claim 2, wherein each image of the first training image set is processed according to the software-tracked contour before used as the input for training.

4. The method of claim 1, wherein the first machine learning model is a regression model based on convolutional neural network.

5. The method of claim 4, wherein the first machine learning model is a residual neural network (ResNet) model.

6. A method of training an evaluation model to generate predicted evaluation errors related to the differences between software-generated analysis result and adjusted analysis result, comprising training a second machine learning model with multiple second training data sets, each of the multiple second training data sets comprising at least one difference parameter set as inputs for training, at least one geometric parameter set as inputs for training, and an evaluation result as target for training, wherein:
each of the at least one difference parameter set indicates the differences between software-tracked contour and adjusted contour;
each of the at least one geometric parameter set is calculated based on a software-tracked contour generated by an analysis software; and
the evaluation result is determined based on the differences between a software-generated analysis result and an adjusted analysis result.

7. The method of claim 6, wherein the second machine learning model is a tree-based model.

8. The method of claim 7, wherein the second machine learning model is a regression model, and the evaluation result is an error value indicating the difference between the software-generated analysis result and the adjusted analysis result.

9. The method of claim 7, wherein the second machine learning model is a classification model, and the evaluation result is a class indicating a good quality or bad quality of the software-generated analysis result.

10. The method of claim 6, wherein each of the at least one geometric parameter set is generated by the steps of:
(a) generating, by the analysis software, a software-tracked contour from at least an image; and
(b) calculating one of the at least one geometric parameter sets based on the software-tracked contour.

11. The method of claim 10, wherein the second training image is an echocardiographic image.

12. The method of claim 6, wherein each of the at least one difference parameter set is generated by the steps of:
(a) obtaining a second training image set by selecting at least one image; and
(b) generating, by a difference model, one of the at least one difference parameter set based on the second training image set.

13. The method of claim 6, wherein each of the at least one difference parameter set is generated by the steps of:
(a) generating, by the analysis software, a software-tracked contour from at least one image;
(b) obtaining an adjusted contour; and
(c) calculating one of the at least one difference parameter set based on the software-tracked contour and the adjusted contour.

14. The method of claim 6, wherein the at least one geometric parameter set comprises an ED (end-diastolic) geometric parameter set and an ES (end-systolic) geometric parameter set; and the ED geometric parameter set and the ES geometric parameter set are generated by the steps of:
(a) obtaining an ED training image set by selecting at least one ED image;
(b) obtaining an ES training image set by selecting at least one ES image;
(c) generating, by the analysis software, a tracked ED contour based on the selected at least one ED image
(d) generating, by the analysis software, a tracked ES contour based on the selected at least one ES image;
(e) calculating the ED geometric parameter set based on the tracked ED contour; and
(f) calculating the ES geometric parameter set based on the tracked ES contour.

15. The method of claim 14, wherein the at least one difference parameter set comprises an ED (end-diastolic) difference parameter set and an ES (end-systolic) difference parameter set, and the ED difference parameter set and the ES difference parameter set are generated by the steps of:
(a) generating, by an ED difference model, the ED difference parameter set based on the ED training image set; and
(b) generating, by an ES difference model, the ES difference parameter set based on the ES training image set.

16. The method of claim 14, wherein the at least one difference parameter set comprises an ED (end-diastolic) difference parameter set and an ES (end-systolic) difference parameter set, and the ED difference parameter set and the ES difference parameter set are generated by the steps of:
(a) obtaining an adjusted ED contour and an adjusted ES contour;
(b) calculating the ED difference parameter set based on the tracked ED contour and the adjusted ED contour; and
(c) calculating the ES difference parameter set based on the tracked ES contour and the adjusted ES contour.

17. The method of claim 14, wherein the evaluation result is generated by the steps of:
(a) calculating a software-generated analysis result based on the tracked ED contour and the tracked ES contour;
(b) obtaining an adjusted ED contour and an adjusted ES contour;
(c) calculating an adjusted analysis result based on the adjusted ED contour and the adjusted ES contour;
(d) determining the evaluation result based on the software-generated analysis result and the adjusted analysis result.

18. A method of quality control for software-analyzed images, comprising:
(a) receiving at least one input image and at least one corresponding software-analyzed image, wherein the at least one corresponding software-analyzed image is generated by analyzing the at least one input image with an analysis software;
(b) generating, by at least one difference model, at least one set of predicted difference parameters based on the at least one input image;
(c) generating at least one set of geometric parameters from the at least one corresponding software-analyzed image; and
(d) generating, by an evaluation model, a predicted evaluation result based on the at least one set of predicted difference parameters and the at least one set of geometric parameters.

19. The method of claim 18, before generating the at least one set of first difference parameters further comprising:
processing the at least one input image based on the at least one software-analyzed input image.

20. The method of claim 18, wherein the input image is an echocardiographic image.

21. The method of claim 18, wherein the evaluation result is an error value indicating the difference between a software-generated analysis result and an adjusted analysis result.

22. The method of claim 18, wherein the evaluation result is a class indicating a good quality or bad quality of the software-generated analysis result.

23. The method of claim 18, wherein the at least one corresponding software-analyzed image includes a software-tracked contour.

24. The method of claim 18, wherein:
the at least one input image comprises at least one ED input image and at least one ES input image;
the at least one corresponding software-analyzed image comprises an ED corresponding image and an ES corresponding image;
the at least one difference model comprises an ED difference model and an ES difference model;
the at least one set of predicted difference parameters comprises one set of predicted ED difference parameters and one set of predicted ES difference parameters; and
the at least one set of geometric parameters comprises one set of ED geometric parameters and one set of ES geometric parameters.

25. A non-transitory computer-readable medium having stored thereon a set of instructions that are executable by a processor of a computer system to carry out a method of:
(a) receiving at least one input image and at least one corresponding software-analyzed image, wherein the at least one corresponding software-analyzed image is generated by analyzing the at least one input image with an analysis software;
(b) generating, by at least one difference model, at least one set of predicted difference parameters based on the at least one input image;
(c) generating at least one set of geometric parameters from the at least one corresponding software-analyzed image; and
(d) generating, by an evaluation model, a predicted evaluation result based on the at least one set of predicted difference parameters and the at least one set of geometric parameters.
